(19) United States
(12) Patent Application Publication
(10) Pub. No.: US 2016/0067083 A1
(43) Pub. Date: Mar. 10, 2016

(54) CANNULA INSERTION SUSTAINING SYSTEMS AND DEVICES

(71) Applicant: Doheny Eye Institute, Los Angeles, CA (US)

(72) Inventors: Jaw-Chyng Lormen Lue, San Gabriel, CA (US); Mark Humayun, Glendale, CA (US)

(21) Appl. No.: 14/796,453

(22) Filed: Jul. 10, 2015

Related U.S. Application Data
(60) Provisional application No. 62/047,210, filed on Sep. 8, 2014.

(51) Int. Cl. A61F 9/00

(52) U.S. Cl. CPC A61F 9/0017

(57) ABSTRACT

A cannula device for an ophthalmic surgical procedure includes a cannula configured to be inserted through a sclera of an eye and a disk structure configured to apply a force on the sclera to sustain a position of the cannula. A cannula insertion sustaining system for an ophthalmic procedure includes a cannula tube configured to be inserted through a sclera of an eye, an upper tube portion, and a connector tube coupled to the upper tube portion and to the cannula tube. The connector tube is configured to reduce an effective pulling vector force asserted on the cannula tube. A method of sustaining a position of a cannula in a sclera includes inserting the cannula through the sclera and engaging a support structure with the sclera to apply a force on the sclera.

### INCORPORATION BY REFERENCE TO ANY PRIORITY APPLICATIONS

The present application claims priority benefit of U.S. Provisional Patent App. No. 62/047,210, filed on Sep. 8, 2014. Any and all applications for which a foreign or domestic priority claim is identified in the Application Data Sheet as filed with the present application are hereby incorporated by reference under 37 C.F.R. §1.57.

### BACKGROUND

1. Field

The disclosure relates generally to the field of ophthalmic surgery, and, more particularly, to conducting ophthalmic surgery using one or more cannulas.

2. Description

In ophthalmic surgery, and in other surgical contexts, a surgeon may desire to create a port opening on the surface tissue of a patient and to insert a cannula through which the surgeon can perform surgical operations. Generally, such port openings are called cannulas in the ophthalmic surgery context.

### SUMMARY

Advancements in technology make it possible to perform minimally invasive surgery and/or other medical or other procedures, in particular ophthalmic surgery, by use of one or more cannula systems.

In some embodiments, a cannula device for an ophthalmic surgical procedure comprises, or alternatively consists essentially of, a cannula configured to be inserted through a sclera of an eye and a disk structure configured to apply a force on the sclera to sustain a position of the cannula.

The force may comprise at least one of surface tension forces, suction forces, adhesion forces, and vacuum forces. The disk structure may comprise a circular disk structure configured to fit a curvature of an eye. The disk structure may comprise a circular disk structure curved more than a curvature of an eye. The disk structure may comprise a rim pattern. The rim pattern may comprise a plurality of protrusions that extend radially outward from an arcuate portion of the disk structure. The rim pattern may comprise a surface portion at an outer tip portion of the plurality of protrusions. The cannula device may further comprise a cannula body portion configured to be above a sclera of an eye. The cannula body portion may comprise a plunger structure configured to create a vacuum force between the disk structure and a sclera. The cannula device may further comprise a bellow portion configured to generate a vacuum force between the disk structure and a sclera. The cannula device may further comprise a stepped cup configured to generate a vacuum force between the disk structure and a sclera. The cannula device may further comprise a vacuum release apparatus. A cannula insertion sustaining system for an ophthalmic procedure may comprise the cannula device and a connector tube coupled to the cannula. The connector tube may be configured to reduce an effective pulling force on the cannula.

In some embodiments, a cannula insertion sustaining system for an ophthalmic procedure comprises, or alternatively consists essentially of, a cannula tube configured to be inserted through a sclera of an eye, an upper tube portion, and a connector tube coupled to the upper tube portion and to the cannula tube. The connector tube is configured to reduce an effective pulling vector force asserted on the cannula tube.

The connector tube may be flexible to enable the connector tube to be curved. The connector tube may be flexible to enable the connector tube to be curved up to an angle. The connector tube may be curved at an angle. The cannula insertion sustaining system may further comprise a plunger structure configured to create a vacuum force for sustaining a position of the cannula. The cannula insertion sustaining system may further comprise a support structure configured to apply a force on the sclera. The force may be configured to enable the support structure to sustain a position of the cannula tube. The force may comprise at least one of surface tension forces, suction forces, adhesion forces, and vacuum forces. The support structure may comprise a disk structure.

In some embodiments, a method of sustaining a position of a cannula in a sclera comprises, or alternatively consists essentially of, inserting the cannula through the sclera and engaging a support structure with the sclera to apply a force on the sclera.

Inserting the cannula through the sclera may comprise inserting a trocar through the sclera. The cannula may be around the trocar. The method may further comprise actuating a plunger structure to create a vacuum between the support structure and the sclera. The method may further comprise actuating a bellow portion to create a vacuum between the support structure and the sclera. The support structure may comprise a vacuum release apparatus. The method may comprise engaging the vacuum release apparatus to release at least part of the force on the sclera. The method may further comprise inserting a fluid through the cannula. The cannula may be coupled to a tube connector. The method may comprise applying a pulling force to the cannula. The tube connector may reduce an effect of the pulling force on the cannula.

In some embodiments, a cannula insertion sustaining system comprises, or alternatively consists essentially of, a disk structure configured to provide suction force, adhesion, and surface tension to sustain a position of a cannula inserted into a sclera, bent infusion tip connector configured to divert at least a portion of accidental pulling force exerted on the cannula system, and a plunger structure configured to further provide suction force to sustain the position of the cannula inserted into the sclera.

In some embodiments, a cannula insertion sustaining system comprises, or alternatively consists essentially of, a cannula, a support structure, an upper tube portion, and a connector tube. The cannula is configured to be inserted through a sclera of an eye. The support structure is configured to apply a force on the sclera. The support structure is configured to be coupled to the cannula. The force is configured to enable the support structure to sustain a position of the cannula in the sclera. The connector tube is configured to couple to the upper tube portion to the cannula. The connector tube is configured to be flexible to enable the connector tube to curve. The curving of the connector tube is configured to reduce an effective pulling vector force asserted on the cannula.

The support structure may comprise a disk structure having a rim pattern. The rim pattern may comprise a plurality of protrusions that extend radially from a perimeter of the disk structure. The plurality of protrusions may comprise surface portions at outer tip portions of the plurality of protrusions. The support structure may comprise a circular disk structure configured to fit a curvature of an eye. The support structure may comprise a wall thickness of less than about 0.6 mm. The cannula insertion sustaining system may further comprise a plunger structure. The plunger structure may be integrally formed with the support structure. The plunger structure may be configured to create a vacuum force for sustaining the position of the cannula. The plunger structure may comprise a stepped-in configuration that is integrally formed with the support structure. The stepped-in configuration of the plunger structure may be configured to create a vacuum force for sustaining the position of the cannula. The plunger structure may comprise a compressible bellow portion. The compressible bellow portion may be configured to generate a vacuum for generating suction with the sclera to maintain the position of the support structure. The compressible bellow portion may be integrally formed with the support structure. A diameter of a widest portion of the bellow portion may be larger than a diameter of a widest portion of the support structure. The connector tube may be curved at a particular degree. The force may comprise surface tension forces. The force may comprise suction forces. The force may comprise adhesion forces. The force may comprise vacuum forces. The force may comprise at least one of surface tension forces, suction forces, adhesion forces, and vacuum forces. The cannula may comprise an outer surface having protrusions for maintaining the position of the cannula in the sclera. The support structure may comprise a vacuum release apparatus. The vacuum release apparatus may comprise a protrusion portion configured for a user to release the force applied by the support structure.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

### BRIEF DESCRIPTION OF THE DRAWINGS

FIG. 1 is a top perspective view of an example cannula insertion sustaining system comprising a bent and/or curved infusion tip or tool connector portion.

FIG. 2A is a side view of an example cannula portion comprising a circular disk skirt that is curved to the human sclera.

FIG. 2B is a top perspective view of the example cannula portion of FIG. 2A.

FIG. 3A is a side view of an example cannula portion comprising a circular disk skirt that is curved more than the curve of a human sclera.

FIG. 3B is a top perspective view of the example cannula portion of FIG. 3A.

FIG. 4A is a side view of an example cannula portion comprising a circular plunger-like structure.

FIG. 4B is a top perspective view of the example cannula portion of FIG. 4A.

FIG. 5A is a top perspective view of an example cannula portion comprising an example patterned rim.

FIG. 5B is a top perspective view of an example cannula portion comprising another example patterned rim.

FIG. 6A is a bottom perspective view of an example implementation of an example cannula insertion sustaining system.

FIGS. 6B and 6C illustrate an example of an application of a cannula insertion sustaining system on a porcine eye.

FIGS. 6D and 6E illustrate an example test of adhesion and surface tension forces of an example cannula insertion sustaining system.

FIG. 6F provides two graphs of weight change histograms showing retaining force of the pseudo cannula insertion sustaining system of FIGS. 6D and 6E as a function of time.

FIG. 7A is a side view of an example cannula portion comprising a bellow portion.

FIG. 7B is a top perspective view of the example cannula portion of FIG. 7A.

FIG. 8 is a top perspective view an example of a cannula portion comprising a bellow portion.

FIG. 9 is a top perspective view of an example of a cannula portion comprising a stepped cup portion.

FIG. 10A is a top perspective view of an example of a cannula portion comprising a vacuum release apparatus.

FIG. 10B is a top perspective view of an example of a cannula portion comprising a vacuum release apparatus.

### DETAILED DESCRIPTION

Although several embodiments, examples, and illustrations are disclosed herein, it will be understood by those of ordinary skill in the art that the inventions described herein extends beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise combinations of features, and no single feature may be solely responsible for desirable attributes or essential to practicing the inventions herein described.

With the development of new technologies in ophthalmic surgery, there is an increasing need to conduct minimally invasive surgery. In order to conduct minimally invasive surgery, surgeons and medical professionals often use one or more cannulas in conjunction with a trocar to guide various tools and materials into the surgical area. However, due to movement of the patient, surgeon, and/or other medical professional, and also for other reasons, it can be difficult to maintain an optimal position of a cannula throughout the duration of surgery and/or other medical operation. Accordingly, it can be advantageous to have a system and/or methods for sustaining and/or stabilizing the position of a cannula insertion during an ophthalmic surgical procedure or the like.

The disclosure herein provides certain systems and devices configured to sustain and/or stabilize the position of a cannula insertion to inhibit or prevent the cannula from being accidentally removed during ophthalmic surgery and/or other medical procedures.

During ophthalmic surgery and/or other ophthalmic procedures, one or more trocar needles are often used to puncture one or more holes in the sclera through and/or over which a cannula can be pushed to guide one or more tools into the eye for surgery. Generally, the tension force exerted by the sclera around the circumference of the cannula tube keeps the cannula in place in the sclera to a certain degree. However, as a surgeon and/or other medical professional makes slight adjustments to the angle of the cannula tube during surgery or another medical procedure, either on purpose or by accident, the size of incision in the sclera can become larger. Movement by the patient can also or alternatively increase the size of incision in the sclera. The weight of an infusion tube, tool insertion tube, and/or other component of a cannula system can also or alternatively increase the size of incision in the sclera. As a result of any increase in the size of incision in the sclera, the retention force around the circumference of the cannula tube can become weaker. In turn, the position of the cannula and/or cannula tube may be destabilized and can be pushed (e.g., due to pressure from fluid in the eye) and/or pulled (e.g., from an accidental pulling force) out from the sclera. For infusion cannulas through which fluid such as water, balanced salt solution (BSS), and/or other liquid can be infused into the eye, the eye can become pressurized and force the cannula and/or infusion tube out. Generally, if a cannula is inadvertently removed during surgery, the same and/or different cannula must be inserted and/or stabilized before a surgeon or other medical professional can proceed with the procedure.

Accordingly, it is desirable for an inserted cannula to remain in an intended position during the duration of surgery and/or other medical procedure. As such, certain cannula insertion sustaining systems and devices described herein are configured to support and/or substantially inhibit or prevent a cannula from being accidentally removed from the sclera during ophthalmic surgery and/or other medical procedures. For example, some embodiments of the cannula insertion described herein can inhibit or prevent a cannula from being accidentally pulled off from the surgical area and/or eye via adhesion, surface tension, vacuum force, air pressure from the ambient atmosphere, an elastomeric disk-shaped ring, plunger, and/or configuration of infusion tip connector, among others. Due to the substantially stabilized insertion and position of the cannula by utilizing the cannula insertion sustaining system and devices described herein, an ophthalmic surgery and/or other medical procedure can more efficiently be performed without unnecessary delays and complications that can arise from an unstable cannula.

The features described herein in connection with one or more embodiments of the cannula insertion sustaining systems and devices can be utilized for any type or kind of cannula systems currently existing or to be developed in the future. In addition, the example cannula insertion sustaining systems and devices described herein can comprise any of the features described herein individually and/or in combination with one or more features.

Certain embodiments of the cannula insertion sustaining system and devices described herein can be low-profile and/or cost-effective. Certain embodiments of the cannula insertion sustaining systems and devices described herein can be easily produced at a low cost. Partially due to low cost and ease of manufacture, certain embodiments of the cannula insertion sustaining systems and devices described herein can be disposable. The disposable nature of the cannula insertion sustaining systems and devices can further provide or guarantee a high level of personal hygiene as a single device and/or system can be disposed after a single use. Some embodiments of the cannula insertion sustaining systems and devices described herein are compatible with one or more existing cannula systems.

### Infusion Tip Connector

In traditional cannula systems, generally an infusion tip and/or tool connector portion comprises a straight configuration. The infusion tip and/or tool connector portion is configured to be inserted into a cannula tube in order to guide and/or direct an infused material and/or tool. Because of its straight configuration, if an accidental pulling force is exerted along the axis of the infusion tip and/or tool connector and away from the surgical area (aligned with a central axis of a cannula insertion tube), substantially all of such pulling force is transferred directly to the cannula system. As a result, there is a high likelihood that such traditional cannula systems can be inadvertently removed from the surgical site when such a pulling force is exerted on the cannula system. Accordingly, it can be desirable to mitigate the effects of such pulling forces that can be exerted on a cannula system.

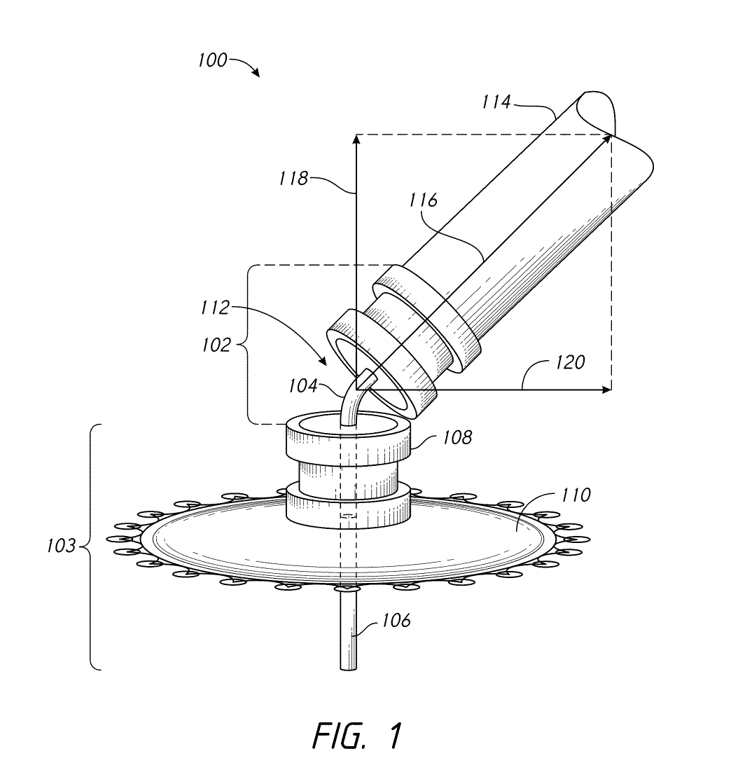

To reduce or mitigate the effects of accidental pulling forces on a cannula system, in some embodiments of a cannula insertion sustaining system as described herein, an infusion tip and/or a tool connector portion of a cannula system is bent and/or curved. FIG. 1 is a top perspective view of an example cannula insertion sustaining system 100 comprising a bent and/or curved infusion tip or tool connector portion 102.

As illustrated in FIG. 1, some embodiments of the cannula insertion sustaining systems and devices can comprise a bent and/or curved infusion tip or tool connector portion 102 and a cannula portion or device 103. The infusion tip and/or tool connector portion 102 can comprise an infusion tube and/or tool insertion tube 104 (also referred to herein as a connector tube). The infusion tube and/or tool insertion tube 104 can comprise a hollow tube with thin walls to assist in the infusion and/or insertion of one or more tools or other materials through the sclera and into an eye. The infusion tube and/or tool insertion tube 104 can be configured to be inserted into and/or coupled to a cannula or cannula tube 106, which can be inserted further into an ophthalmic and/or other surgical area. In some embodiments, the cannula tube 106 comprises an outer surface having grooves, protrusions, and/or compositions configured to maintain the cannula tube 106 in the sclera through friction forces. The cannula portion 103 comprises the cannula tube 106, a cannula body portion 108, and optionally a disk structure or support structure 110. In some embodiments, the cannula tube has a diameter less than about 0.5 mm, for example between about 0.1 mm and about 0.5 mm.

In some embodiments, the infusion tube and/or tool insertion tube 104 comprises a bent and/or curved configuration as illustrated in FIG. 1. In such embodiments, when an accidental pulling force is exerted on the cannula system 100 along the central axis of an upper tube portion and away from the surgical area, not all of the accidental pulling force is effective on the cannula system 100 as in traditional cannula systems. Rather, the direction of the accidental pulling force is not aligned with a central axis of a cannula tube 106. The direction of the accidental pulling force can form an angle with the direction of the cannula tube 106 due to the bent and/or curved infusion tube and/or tool insertion tube 104.

In other words, a force vector of the accidental pulling force can comprise two or more orthogonal components as illustrated in FIG. 1 (see force diagram imposed on FIG. 1). More specifically, a diagonal accidental pulling force 116 can comprise a vertical component 118 and a horizontal component 120. The horizontal component 120 can be substantially ineffective to remove the cannula system 100 from its position, because the horizontal component 120 is perpendicular to the direction of removal of the cannula system 100. The vertical component 118, on the other hand, can directly affect the removal of the cannula system 100 as the vertical component 118 is aligned with the direction of the cannula tube 106. However, the vertical component 118 is weaker than the diagonal accidental pulling force 116, as illustrated in FIG. 1 by having a shorter arrow. Accordingly, not all of the accidental pulling force 116 is translated to potentially remove the cannula system 100 from the surgical site, in contrast to traditional cannula systems in which all of an accidental pulling force is translated to potentially remove the cannula system from the surgical site. In other words, only part of the accidental pulling force 116 may be effective along the weakest connection, the vertical component 118 aligned with the direction of the cannula tube 106. As such, a bent and/or curved connector tip 102 configuration can help reduce the risk of accidentally pulling off a cannula system 100 during a surgical and/or other medical procedure.

In some embodiments, the bent and/or curved configuration of an infusion tube or tool insertion tube 104 can reduce or substantially reduce the effective pulling force by about 10%, about 20%, about 30%, about 40%, about 50%, about 60%, about 70%, about 80%, about 90%, about 100%, and/or within a range defined by any of the aforementioned values, for example depending on the angle at which the infusion tip and/or tool connector 102 is bent.

In some embodiments, the angle that is formed between the upper tube portion and the cannula tube 106 due to the bent and/or curved infusion tube and/or tool insertion tube 104 can be about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, about 90 degrees, about 95 degrees, about 100 degrees, about 105 degrees, about 110 degrees, about 115 degrees, about 120 degrees, about 125 degrees, about 130 degrees, about 135 degrees, about 140 degrees, about 145 degrees, about 150 degrees, about 155 degrees, about 160 degrees, about 165 degrees, about 170 degrees, about 175 degrees, about 179 degrees, and/or in a range defined by any two of the values listed above.

In some embodiments, the infusion tube and/or tool insertion tube 104 comprises a flexible, elastic, and/or elastomeric material configured to allow the infusion tube and/or tool insertion tube 104 to be bent and/or curved. In some embodiments, the infusion tube and/or tool insertion tube 104 comprises a metal, alloy, and/or plastic material.

In some embodiments, the infusion tip and/or tool connector portion 102 can be configured to be bent and/or curved prior to, during, and/or after a surgical or other medical procedure. In some embodiments, the infusion tip and/or tool connector portion 102 is pre-bent and/or curved during the production stages to a particular degree. In some embodiments, the connector tube portion 102 is non-flexible and is curved or bent at a particular degree or angle. In some embodiments, the connector tube portion 102 is flexible enough to curve or bend to a particular degree or angle, but not further.

In some embodiments, the infusion tip and/or tool connector portion 102 comprises a gap 112, as illustrated in FIG. 1. In certain embodiments, if the infusion tube and/or tool insertion tube 104 comprises a flexible and/or elastic material and, if the infusion tip and/or tool connector portion 102 comprises a gap 112, the upper tube portion 114 of a cannula system can be configured to wobble or otherwise move. In certain embodiments, the upper tube portion 114 of the cannula system 100 can wobble or otherwise move until the weight of the upper tube portion 114 contacts another surface to stabilize or stop movement. For example, the upper tube portion 114 can contact one or more surfaces of a cannula body portion 108, the body of a patient, a cloth surrounding the eye of the patient, and/or other surfaces.

In some embodiments, the infusion tip and/or tool connector portion 102 is completely covered by a casing. The casing can comprise a flexible and/or elastic material that can be bent and/or curved during a surgical procedure and/or other medical procedure. In other embodiments, the casing can comprise a plastic and/or a metallic material that is pre-formed in a curved and/or bent configuration.

In some embodiments, an infusion tip and/or tool connector portion 102 is color coded. In some embodiments, a cannula body portion 108 is also color coded. In certain such embodiments, a surgeon and/or other medical professional can easily match a cannula body portion 108 with an appropriate infusion tip and/or tool connection portion 102. For example, the cannula body portion 108 and the infusion tip and/or tool connection portion 102 can be color coded according to the gauge size and/or outer diameter of the cannula body portion 108. More specifically, a 27 gauge (0.4128 mm outer diameter) cannula body portion 108 and infusion tip and/or tool connection portion 102 can comprise a first color and a 25 gauge (0.5144 mm outer diameter) cannula body portion 108 and infusion tip and/or tool connection portion 102 can comprise a second color different than the first color. By color coding cannula body portion 108 and infusion tip and/or tool connection portion 102 according to gauge size, a surgeon and/or other medical professional can easily identify the correct cannula body portion 108 to be used in conjunction with a particular infusion tip and/or tool connection portion 102, or vice versa.

### Disk Structure

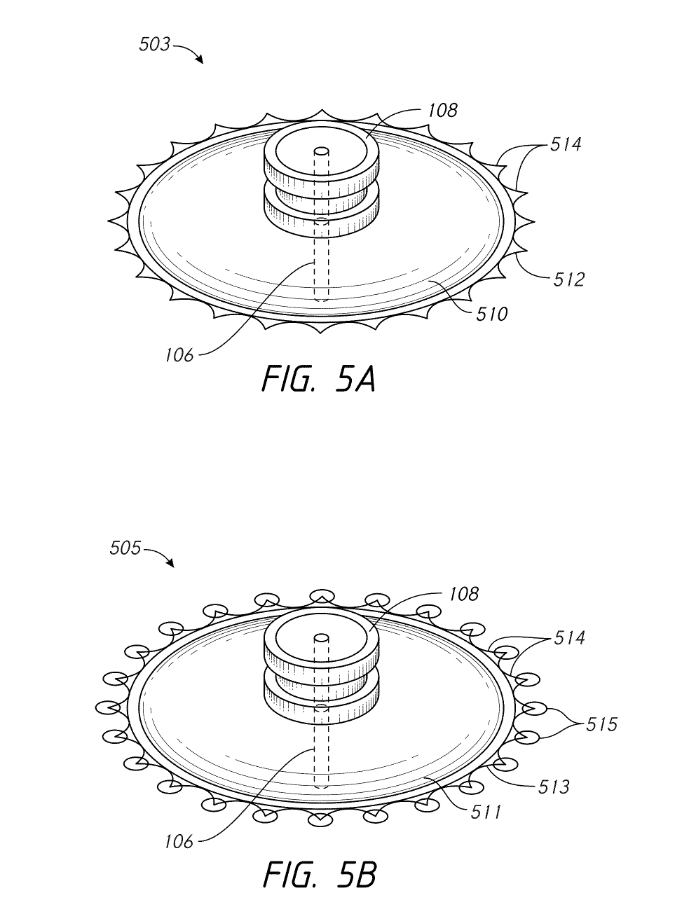

Still referring to FIG. 1, in some embodiments a cannula insertion sustaining system 100 comprises a disk structure or support structure 110. The system 100 comprises a disk structure 110 comprising a particular rim pattern, discussed in further detail herein, for example with respect to FIGS. 5A and 5B.

Some embodiments of the cannula insertion sustaining system 100 optionally comprise a disk structure 110, which may assist with stabilizing and/or sustaining the position of the inserted cannula throughout a duration (e.g., an entire duration) of an ophthalmic and/or other medical procedure. Systems without or lacking a disk structure 110 may rely partially, primarily, or totally on the tension force exerted by the sclera around the circumference of the cannula tube 106 to maintain the position of the cannula tube 106. In systems comprising a disk structure 110, adhesion, surface tension, vacuum, and/or suction force(s) can be provided to assist with stabilizing and/or sustaining the position of an inserted cannula tube 106.

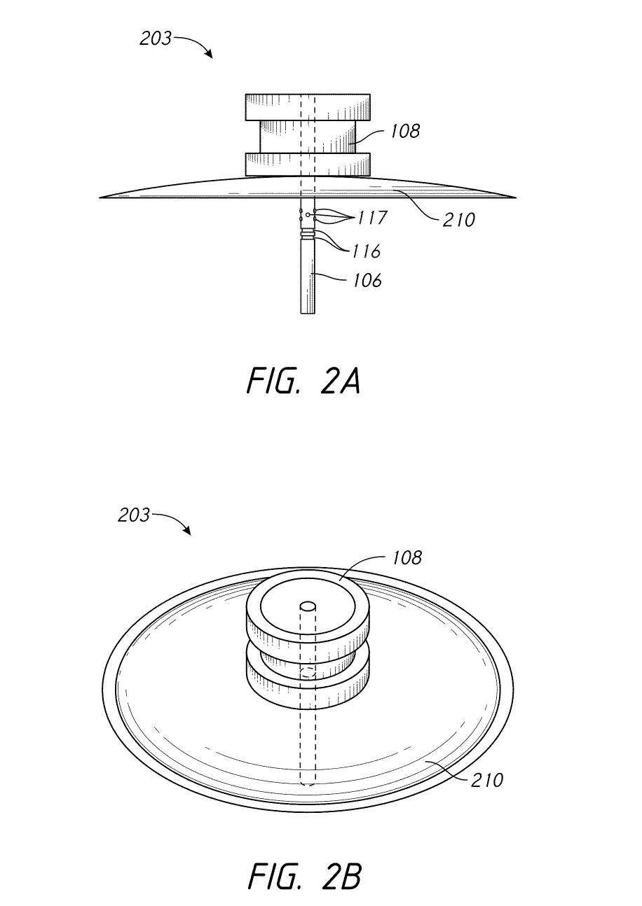

FIG. 2A is a side view of an example cannula portion or device 203, for example for use with a cannula insertion sustaining system (e.g., the system 100), comprising a disk structure 210 in the form of a circular disk skirt that is curved to the human sclera. FIG. 2B is a top perspective view of the example cannula portion 203 of FIG. 2A. The disk structure 210 can be attached to the base or distal end of the cannula body portion 108 and/or along the length of the cannula tube 106. In some embodiments, a cannula body portion 108 can be on top of (e.g., touching or spaced from) the disk structure 210.

In some embodiments, the disk structure 210 comprises a slight curvature to fit (e.g., correspond to) the curvature of a sclera. For example, a concave curvature of a disk structure 210 can be substantially equal to the convex curvature of a normal or standard human sclera (e.g., having a radius of curvature between about 11 mm and about 13 mm (e.g., about 12 mm)). In some embodiments, curvature of the sclera may be measured prior to a procedure, and a disk structure 210 having a curvature to match or exceed that curvature may be selected. When in a stationary position, substantially all portions of the disk structure 210 can be in contact with the human sclera. As tears and/or other body fluid is excreted from the eye, adhesion and surface tension is generated between the disk structure 210 and the human sclera under the disk structure 210. Because all or substantially all of the disk structure 210 is in contact with the sclera, the generated adhesion and surface tension can be applied throughout the whole disk structure 210, thereby functioning as a maintaining force for the cannula tube 106.

Such adhesion and surface tension forces can exist naturally between the two surfaces. In some embodiments, no additional adhesives are used to provide the adhesion and surface tension. That is, the cannula portion 203 may be free of or lacking adhesive. As the area under the disk structure 210 becomes drier, the adhesion and surface tension forces may become even greater due to the higher viscosity of the body fluid between the disk structure 210 and the sclera. For example, a tear can contain mucous, electrolyte, water, and oil, among others.

In some embodiments, when a pulling force is exerted on the cannula portion 203 including the disk structure 210, a vacuum or a degree thereof can be generated under the disk structure 210. In other words, when the cannula portion 203 is pulled in a direction away from the eye, the pressure level under the disk structure 210 can be lower than atmospheric pressure surrounding the exterior of the disk structure 210. As a result of the air pressure difference between the outside and under of the circular disk structure 210, a suction force can be induced, thereby further strengthening the retaining force for securing the position of the cannula portion 210 in the sclerotomy. Since the vacuum force is induced by the air pressure difference between the outside and under the disk structure 210, the more the pulling force, the stronger the reactive suction force will be due to the greater difference in air pressure.

Adhesion and surface tension due to body fluids can continue to be present on surfaces and edges of the disk structure 210, which continues to be in contact with the sclera, including but not limited to the edges of the disk structure 210. When a pulling force is exerted on the cannula portion 203, adhesion between the disk structure 210 and the human sclera, the surface tension along the edges of the disk structure 210 and the suction force generated by vacuum underneath the disk structure 210 can, in combination, can provide retention force in order to secure the position of the cannula and prevent accidental removal thereof. In contrast, when no pulling force is exerted, adhesion and surface tension alone can provide retention force in such embodiments. As more pulling force is exerted, more vacuum and/or suction force can provide retention force, while less adhesion may be present.

In some embodiments, the disk structure 210 comprises an elastomeric and/or flexible material. The disk structure 210 can comprise a variety of sizes such that a disk structure 210 comprising a particular size can be selected and used depending on the surgical procedure and/or a surgical area. For example, the diameter of the disk structure 210 can be about 1 mm, about 2 mm, about 3 mm, about 4 mm, about 5 mm, about 6 mm, about 7 mm, about 8 mm, about 9 mm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, and/or between a range defined by any two of the values listed above. Depending on the surgical procedure and/or area, a disk structure 210 comprising a relatively large diameter can interfere with the procedure, although the disk structure 210 may desirably have a sufficiently large diameter to provide retention force above a certain degree. A user can select a device comprising an appropriately sized disk structure 210 for the particular application.

In some embodiments, the outer surface of the cannula tube 106 comprises a material having a texture configured to increase retention force. For example, the outer surface of a cannula tube 106 that is configured to be inserted into the sclera can comprise radially inward grooves 116, radially outward protrusions 117, combinations thereof, and/or or other compositions. Such compositions can increase the retention force and assist in sustaining the position of the cannula tube 106 and inhibiting or preventing accidental removal thereof.

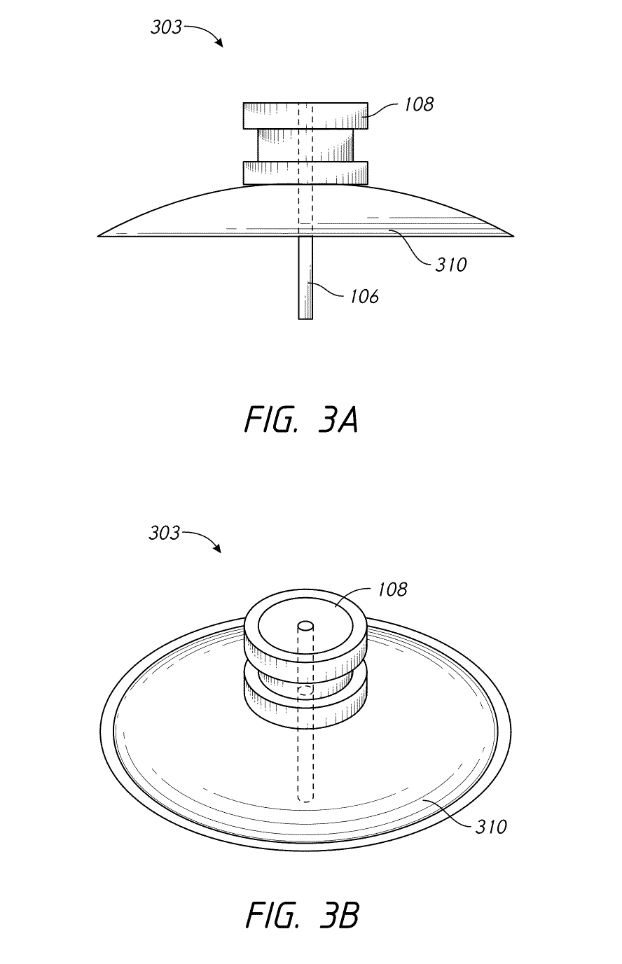

FIG. 3A is a side view of an example cannula portion or device 303, for example for use with a cannula insertion sustaining system (e.g., the system 100), comprising a circular disk skirt 310 that is curved more than the curve of a human sclera. FIG. 3B is a top perspective view of the example cannula portion 303 of FIG. 3A. The disk structure 310 can be attached to the base or distal end of the cannula body portion 108 and/or along the length of the cannula tube 106. In some embodiments, a cannula body portion 108 can be on top of (e.g., touching or spaced from) the disk structure 310.

In some embodiments, the disk structure 310 comprises a curvature that is curved more than the curvature of a normal or standard human sclera (e.g., having a radius of curvature between about 12 mm and about 20 mm (e.g., about 15 mm)). The greater curvature of the disk structure 310 can provide an area near the center of the circular disk skirt 310 and under the circular disk skirt 310 that will be spaced from or not in contact with the human sclera by a gap. As a result, the area and/or force of adhesion in such embodiments can be less than for a disk structure such as, for example, the disk structure 210 described herein, but the gap and/or vacuum under the central portion of the disk structure 310 can provide suction force. As described herein, the vacuum that is present under the disk structure 310, or between the disk structure 310 and the sclera, or in the gap, can provide a suction force that can assist in stabilizing and/or sustaining the position of an inserted cannula tube 106.

As described above with respect to the disk structure 210, when a pulling force is exerted on a cannula system, an even greater vacuum and/or suction force will be induced under the disk structure 310 due to the greater pressure difference between the outside and the gap under the disk structure 310. As a result, the more the pulling force, the stronger the reactive suction force will be due to the greater difference in air pressure.

Despite the greater curvature of the disk structure 310, the edges of the disk structure 310 will be in contact with the human sclera. As such, surface tension can still be present around the rim of the disk structure 310 due to excretion of body fluids. The surface tension that is generated along the edges of the disk structure 310 can provide a retention force to sustain the position of an inserted cannula tube 106.

In some embodiments, a ratio between an adhesion force and a vacuum and/or suction force provided by a disk structure (e.g., the disk structure 310) can be controlled and/or manipulated by varying the curvature of the disk structure. For example, by increasing the curvature, the volume under a disk structure can be increased to increase vacuum and/or suction force. By decreasing the curvature, the volume under a disk structure can be decreased to decrease vacuum and/or suction force. The amount of area of a disk structure 310 that is or will be in contact with a sclera can also be controlled by varying the curvature.

A disk structure may reduce or eliminate other structures intended to maintain a position of a cannula tube, such as barbs, hooks, and the like, which are generally difficult to remove and may cause tearing, inflammation, and/or other issues. In some embodiments, a cannula insertion sustaining system or a cannula portion is free of, substantially free of, lacking, or without means for physically engaging a sclera or other body tissue below the outer surface of the body tissue such as barbs or hoods.

### Plunger Structure

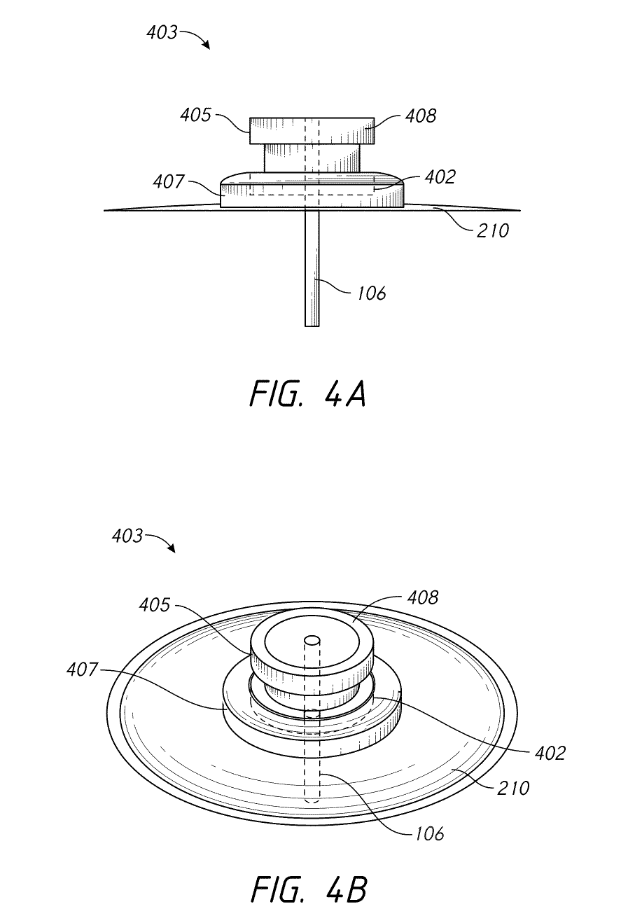

FIG. 4A is a side view of an example cannula portion or device 403, for example for use with a cannula insertion sustaining system (e.g., the system 100), comprising a circular plunger-like structure. FIG. 4B is a top perspective view of the example cannula portion 403 of FIG. 4A. The disk structure 210 can be attached to the base or distal end of the cannula body portion 408 and/or along the length of the cannula tube 106. In some embodiments, a cannula body portion 408 can be on top of (e.g., touching or spaced from) the disk structure 210. In some embodiments, the disk structure 210 is in the form of a circular disk skirt that is curved to the human sclera.

In some embodiments, a cannula portion 403 comprises a plunger structure 402. The plunger structure 402 may be located under the cannula body portion 408, along the cannula body portion 408, above the disk structure 210, etc. The cannula body portion 408 may comprise a feature, for example the upper disk 405, that is movable relative to the plunger structure 402 (e.g., illustrated as formed in the lower disk 407 in FIGS. 4A and 4B). The upper disk 405 may be coupled to the cannula tube 106 and the plunger structure 402 may be movable relative to the cannula tube 106. In use, the upper disk 405 may be pressed at least partially into the plunger structure 402, forming a vacuum and/or suction pressure between the upper disk 405 and the plunger structure 402. When a pulling force is exerted on a cannula system, an even greater vacuum and/or suction force will be induced between the upper disk 405 and the plunger structure 402. As a result, the more the pulling force, the stronger the reactive suction force will be. This vacuum and/or suction force is separate from, and may be in addition to, a vacuum and/or suction force between the disk structure 210 and the sclera.

In some embodiments, the plunger structure 402 forms a unitary structure with support structure or is formed integrally with the support structure. In some embodiments, the plunger structure 402 is formed separately from the support structure, such that the plunger structure 402 can be coupled to the support structure. The plunger structure 402 can increase a retaining force in addition to the retention force generated from the holding force of the sclerotomy.

In some embodiments, portions of the disk structure 210 and/or the edges of the disk structure 210 can be in contact with the human sclera when in a stationary position. As such, adhesion and surface tension can be present in those areas due to excretion of body fluids. Such generated adhesion and surface tension can provide a retention force that can stabilize and/or sustain the position of an inserted cannula tube 106.

In some embodiments, the disk structure 210 under the plunger structure 402 does not or substantially does not come into contact with the human sclera. In certain such embodiments, even when in a stationary position, a gap and/or vacuum may be present under the plunger structure 402, which can provide a suction force that can stabilize the position of an inserted cannula tube 106.

When the cannula tube 106 is pulled, some portions of the disk structure 210 can cease to be in contact with the human sclera, generating an additional gap and/or vacuum between the disk structure 210 and the human sclera. As described herein, such a vacuum can further provide a suction force that can stabilize the position of an inserted cannula tube 106. When the cannula tube 106 is pulled, the volume under the plunger-like structure 402 can also increase, providing more vacuum force. Such additional vacuum force due to the plunger structure 402 can provide additional suction force to even further increase a retention force for stabilizing the position of an inserted cannula tube 106.

A height of the plunger structure 402 can vary. For example, in some embodiments, the plunger structure 402 may have a height that is longer or shorter and/or a cross-sectional area that is larger or smaller. Generally, embodiments comprising a plunger structure 402 having a longer height and/or larger cross-sectional area can provide stronger vacuum and/or suction forces due to greater volume deformation capability that can induce greater pressure difference between the ambient and under the plunger structure.

The plunger structure 402 can be used in conjunction with a disk structure having any curvature. For example, a cannula insertion sustaining system can comprise a plunger structure 402 and a disk structure that is substantially curved to the human sclera (e.g., the disk structure 210 described herein). For another example, a cannula insertion sustaining system can comprise a plunger structure 402 and a disk structure that is that is curved more than the curvature of a human sclera (e.g., the disk structure 310 described herein). The size or surface area of a disk structure in embodiments of a cannula portion 403 comprising a plunger structure 402 can also vary, for example according to the dimensions described herein.

### Disk Structure Edges

Generally, increasing the surface area that is contact with a sclera increases generated adhesive force, increasing retention force against accidental pulling forces of a cannula system. Increasing the total length or circumference or area of contacting edges of the object to the fluid increases generated surface tension, increasing retention force against accidental pulling forces of a cannula system. In some embodiments, a disk structure of a cannula system comprises edges having a particular shape and/or configuration configured to increase adhesive forces and/or surface tension.

FIG. 5A is a top perspective view of an example cannula portion or device 503, for example for use with a cannula insertion sustaining system (e.g., the system 100), comprising an example patterned rim 512 of a disc structure 510. The rim 512 can increase adhesive forces and/or surface tension versus, for example, an arcuate or purely round edge. The rim 512 and/or edges of the disk structure 510 comprise(s) one or more protrusions 514. In some embodiments, the disk structure 510 comprises a plurality of protrusions 514 that extend radially outwardly from an arcuate perimeter of the disk structure 510. In some embodiments, the protrusions 514 comprise an angular outer tip portion. Such protrusions 514 can increase the length or circumference of the outer perimeter of the disk structure 510 and/or the surface area of the disk structure 510. When a pulling force is exerted, a greater surface tension force can be provided along the perimeter of the disk structure 510 due to the increased length and contact of the protrusions 514 of the disk structure 510 with the sclera. In some embodiments, the increased surface area of the disk structure 510 due to the patterned rim 512 can increase adhesion force, even when the disk structure 510 is stationary. By adding one or more small protrusions 514 along the outer perimeter of the disk structure 510, surface tension can be increased without excessively comprising an area surrounding the disk structure 510. Surgical and/or other medical procedures can be less affected despite the larger surface tension.

FIG. 5B is a top perspective view of an example cannula portion 505 comprising another example patterned rim 513. In comparison to the patterned rim 512, the disk structure 511 comprises the protrusions 514 and further comprises circles and/or surface portions 515 that are attached to and/or integrally formed with the protrusions 514. In some embodiments, the surface portions 515 are positioned at outer tip portions of the protrusions 514. The circles and/or surface portions 515 can further increase the surface area and the length or circumference of the outer perimeter of the disk structure 511 compared to the disk structure 510, which can provide even greater adhesion and surface tension forces along the edges of the disk structure 511 and/or in other areas of contact between the disk structure 511 and the sclera.

### Application of a Cannula Insertion Sustaining System

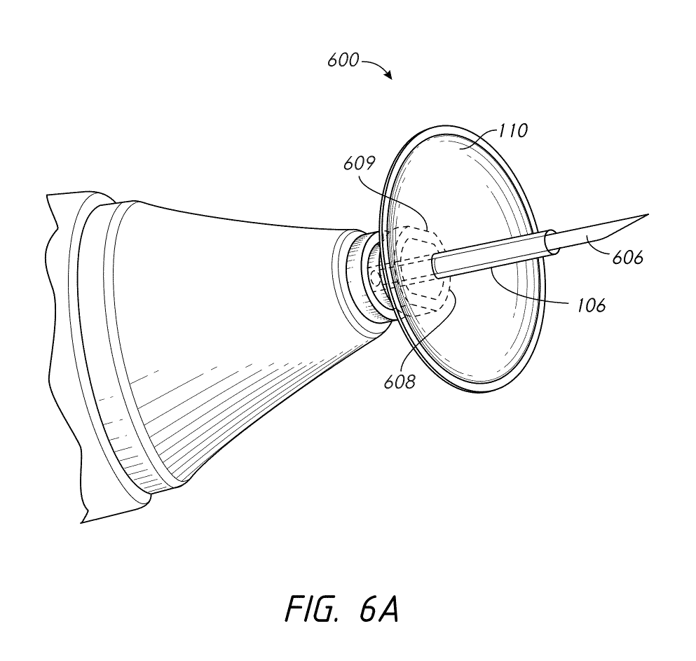

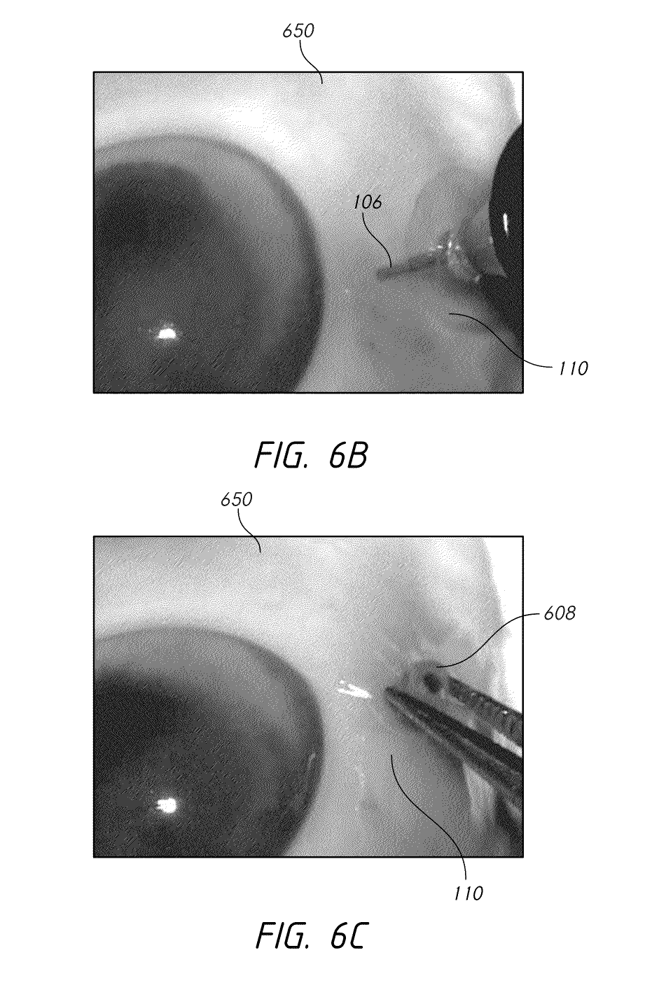

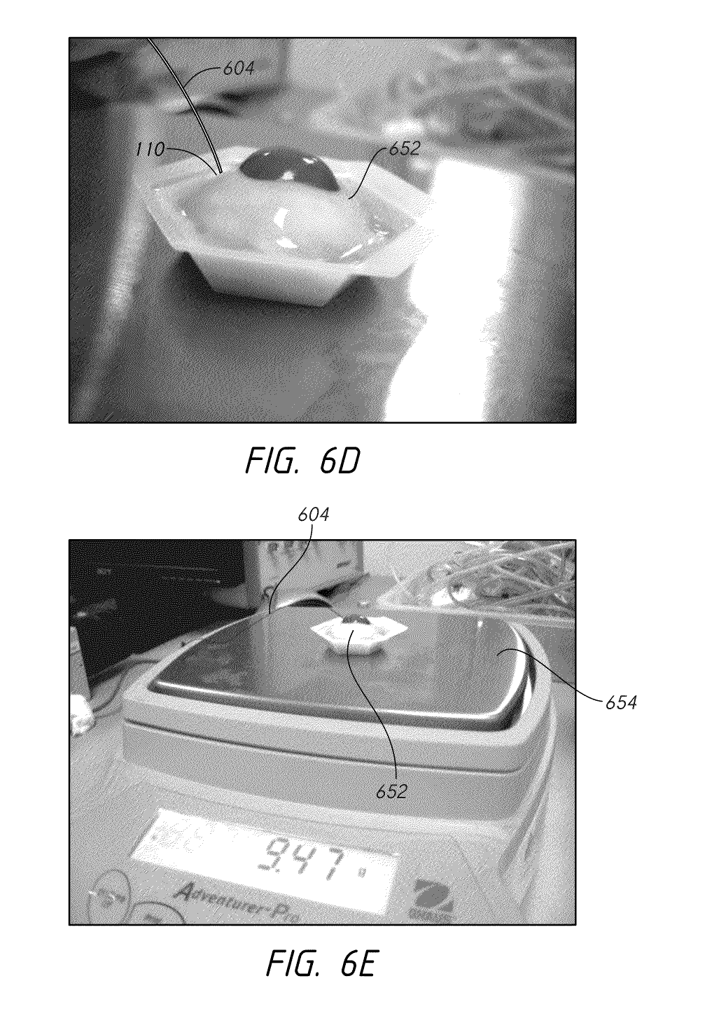

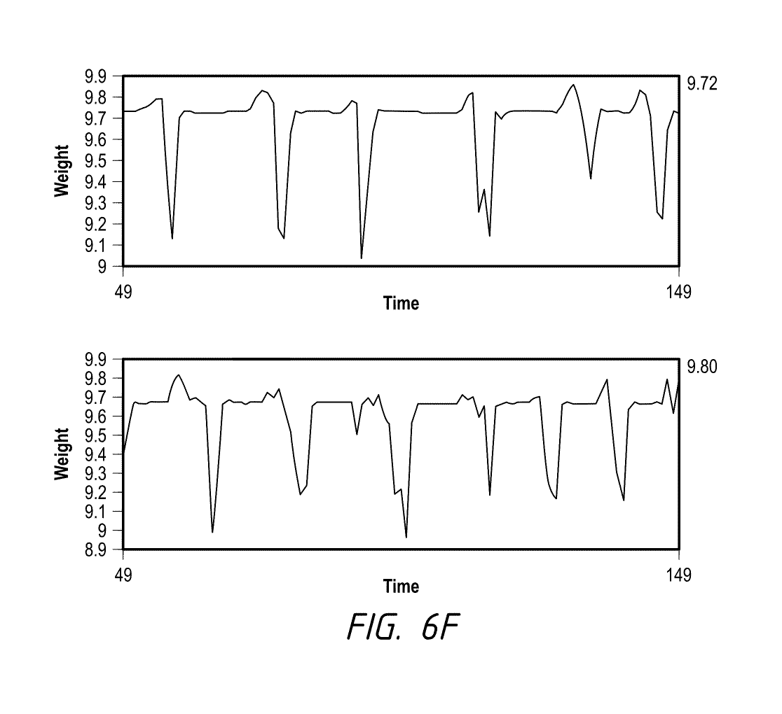

FIG. 6A is a bottom perspective view of an example implementation of an example cannula insertion sustaining system 600. The cannula insertion sustaining system 600 comprises a cannula insertion tube 106, a disk structure 110, a cannula body portion 608, and a trocar or needle 606. The cannula body portion 608 comprises flat surfaces 609, which may provide easier engagement with a cannula moving or removing device such as tweezers. The features of the cannula insertion sustaining system 600 can be modified or substituted as described herein (e.g., alternative disk structures 510, 511, alternative cannula body portion 108, etc.).

FIGS. 6B and 6C illustrate an example of an application of a cannula insertion sustaining system (e.g., the cannula insertion sustaining system 600) on a porcine eye 650. As illustrated in FIG. 6B, the cannula tube 106 is inserted into a porcine eye 650. With the cannula tube 106 inserted, the disk portion 110 is positioned above the sclera of the porcine eye. As illustrated in FIG. 6C, a vacuum chamber can be induced under the disk structure 110 of the cannula insertion sustaining system or between the disk structure 110 and the sclera of the eye 650. As described herein, this generated vacuum chamber can generate a temporary suction force when pulling the cannula body portion 608 away from the sclera.

FIGS. 6D and 6E illustrate an example test of adhesion and surface tension forces of an example cannula insertion sustaining system. The adhesion and surface tension forces may be generated between a disk structure 110 of a cannula insertion sustaining system and a sclera. In the test illustrated in FIGS. 6D and 6E, a pseudo cannula insertion sustaining system is made out of only the disk structure 110 that is glued to a string 604, which represents an infusion tube and/or tool insertion tube such as the tool insertion tube 104. As illustrated in FIG. 6E, a porcine eye 652 was weighed on a balance scale 654 and the weight value was constantly recorded by a computer system as the string 604 was pulled in various strengths.

FIG. 6F provides two graphs of weight change histograms showing retaining force of the pseudo cannula insertion sustaining system of FIGS. 6D and 6E as a function of time. When the string 604 was pulled upward against gravity, the adhesion and surface tension generated by the body fluid between the disk structure 110 and the sclera in addition to the suction force created by the vacuum present under the disk structure 110 stabilized the pseudo cannula system and prevented removal thereof from the eye 652. Instead of the pseudo cannula system simply being removed when a pulling force was exerted, the porcine eye 652 itself was lifted, as shown by the downward peaks of decreased weight. Accordingly, the adhesion, surface tension, and/or vacuum forces generated by the pseudo cannula insertion sustaining system can substantially inhibit or prevent removal of the cannula tube 106 and stabilize the position of the cannula system when a pulling force is exerted thereon. The weight of the string 604, an infusion line filled with water, was about 1.5 grams (g) to about 1.8 g.

In the illustrated test, the retaining force was found to be about 0.7 g. However, depending on the size and/or configuration of a cannula insertion sustaining system, the retaining force can be configured to be about 0.1 g, about 0.2 g, 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, and/or between a range defined by any two of the values listed above.

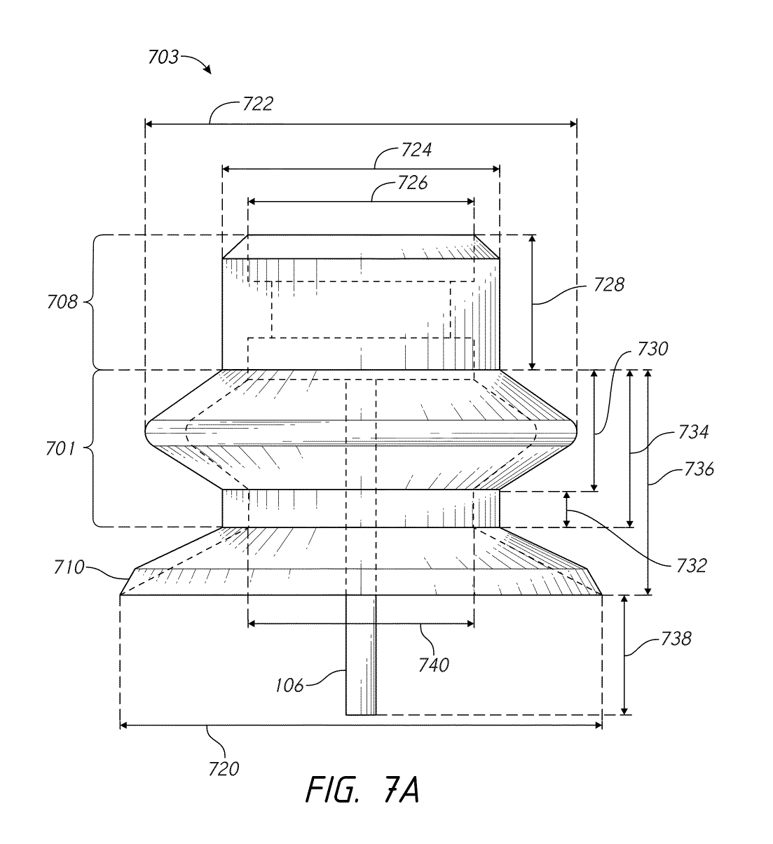

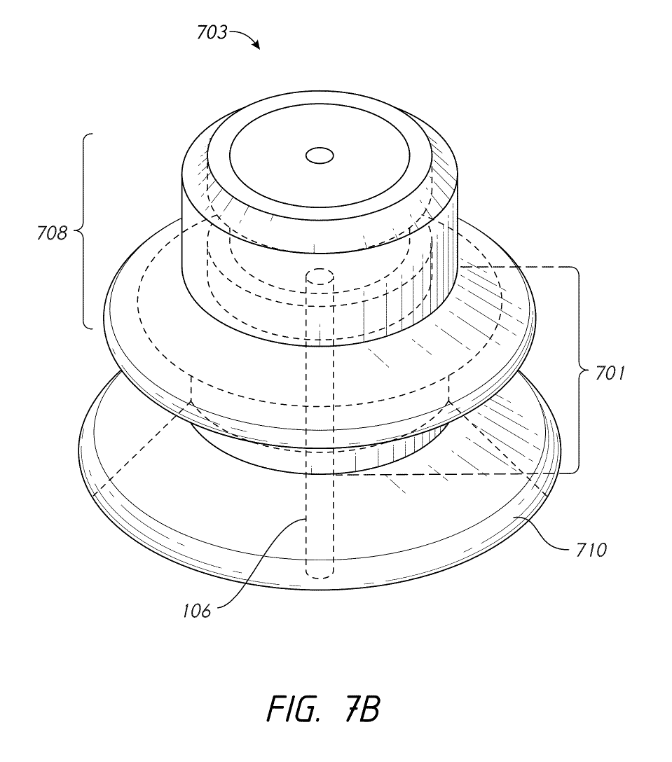

FIG. 7A is a side view of an example cannula portion or device 703, for example for use with a cannula insertion sustaining system (e.g., the system 100), comprising a bellow portion 701. FIG. 7B is a top perspective view of the example cannula portion 703 of FIG. 7A. In some embodiments, the bellow portion 701 is integrally formed with the cannula body portion 708. The bellow portion 701 can comprise, for example, vinyl or other similar material. The cannula body portion 708 may comprise a structure integrated with the structure of the bellow portion 701. In some embodiments, the bellow portion 701 generates suction force and/or provides torque counter balancing sufficient to counteract an accidental pulling force. In some embodiments, the cannula insertion sustaining system comprises a contact lid 710, the portion of the support structure that contacts the sclera, comprising a wall thickness of less than about 0.6 mm. Contact lids 710 comprising a thickness of greater than about 0.6 mm can provide insufficient flexibility for maintaining the shape of the contact surface of the lid 710 in the same or substantially the same shape as the sclera surface. Failure to maintain the shape of the contact surface can generally break the vacuum seal. The mechanical recovering force can be generally sufficiently strong to break the vacuum seal formed by contact lids 710 comprising a thickness of greater than about 0.6 mm. In some embodiments, the cannula insertion sustaining system comprises a bellow portion 701 having a diameter 722 at its widest that is smaller than a diameter 720 of the contact lid 710. A smaller bellow portion 701 can result in a smaller vacuum suction. In some embodiments, a smaller vacuum suction can still hold vacuum when the contact lid 710 is sufficiently thin, for example, about 0.36 mm. The contact lid 710 can conform to the sclera surface and keep its shape while the vacuum maintains a suction hold on the sclera.

Referring to FIG. 7A, the diameter 720 of the contact lid 710 may be between about 4 mm and about 5 mm (e.g., about 0.18 inches or about 4.6 mm), the diameter 722 of the widest part of the bellow portion 701 may be between about 4 mm and about 5 mm (e.g., about 0.175 inches or about 4.4 mm), the diameter 724 of the structure integrated with the structure of the bellow portion 701 may be between about 2.5 mm and about 3.5 mm (e.g., about 0.12 inches or about 3.0 mm), the diameter 726 of the cannula body portion 708 may be between about 2 mm and about 3 mm (e.g., about 0.085 inches or about 2.2 mm), the length or height 728 of the cannula body portion 708 may be between about 1 mm and about 2 mm (e.g., about 0.055 inches or about 1.4 mm), the length or height 730 of a compressible part of the bellow portion 701 may be between about 0.5 mm and about 1.5 mm (e.g., about 0.04 inches or about 1.0 mm), the length or height 732 of another part of the bellow portion 701 may be between about 0.1 mm and about 1 mm (e.g., about 0.02 inches or about 0.5 mm), the length or height 734 of the bellow portion 701 excluding the contact lid 710 may be between about 1 mm and about 2 mm (e.g., about 0.06 inches or about 1.5 mm), the length or height 736 of the bellow portion 701 and the contact lid 710, which may collectively be referenced as a bellow portion 701, may be between about 2 mm and about 3 mm (e.g., about 0.1 inches or about 2.5 mm), the length or height 738 of the cannula tube 106 below the contact lid 710 may be between about 1 mm and about 2 mm (e.g., about 0.06 inches or about 1.5 mm), and/or an inner diameter 740 of a narrowest part of the bellow portion 701 may be between about 1 mm and about 2 mm (e.g., about 0.06 inches or about 1.5 mm). These dimensions are provided as an example only, and may be modified in accordance with the disclosure herein, for example based on an intended use.

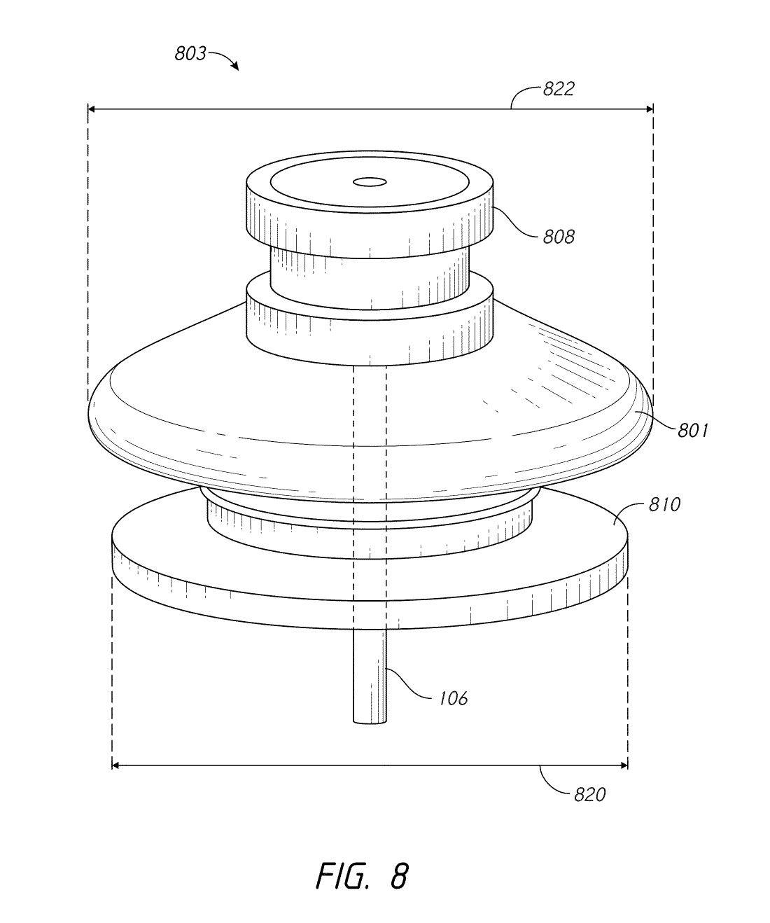

FIG. 8 is a top perspective view an example of a cannula portion or device 803, for example for use with a cannula insertion sustaining system (e.g., the system 100), comprising a bellow portion 801. In some embodiments, the cannula body portion 808 does not comprise a structure integrated with the structure of the bellow portion 801. In some embodiments, the cannula insertion sustaining system comprises a bellow portion 801 having a diameter 822 at its widest that is smaller than a diameter 820 of the contact lid 810. A larger bellow portion 801 can result in a larger vacuum suction.

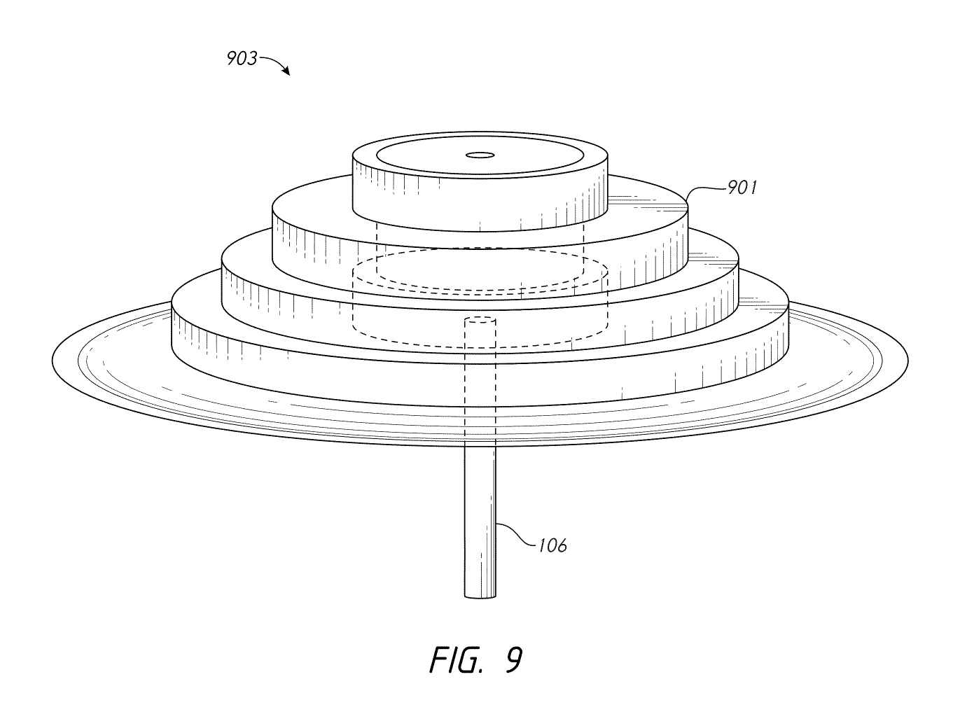

FIG. 9 is a top perspective view of an example of a cannula portion or device 903, for example for use with a cannula insertion sustaining system (e.g., the system 100), comprising a stepped cup portion 901. The stepped cup portion 901 comprises a stepped-in configuration. In some embodiments, the stepped-in configuration is integrally formed with the support structure. In some embodiments, the stepped-in configuration is configured to create a vacuum force for sustaining the position of the cannula tube 106. In some embodiments, the stepped-in configuration is configured to act like a plunger structures or a series of plunger structures, for example as described herein.

In some embodiments, the cannula insertion sustaining system can generate surface tension sufficient to maintain a suction hold on the sclera. In some embodiments, the addition of surface features on the contact lid can reduce the surface tension for maintaining a suction hold on the contact surface. In some embodiments, the cannula insertion sustaining system is limited in size in order to reduce or minimize interference with a surgical or other operation, for example due to limited space at the site of the operation. Due to the size limitations on the cannula insertion sustaining system, a simple curved cup design may not provide a sufficient suction for holding the cannula insertion system in a desired position. In some embodiments, cannula insertion systems comprising a plunger, a stepped-in configuration, and/or a bellow can provide compressible volume sufficient to generate a vacuum that can maintain position of a cannula tube, for example in the presence of a pulling force.

In some embodiments, the general rule is provided by the ideal gas law:

P×V∝n×T, or P∝n×T/V

in which P is pressure, V is volume, n is the number of molecules, and T is temperature.

In some embodiments, the temperature is around room temperature and is constant. In some embodiments, the air molecules can be squeezed out from the compressible volume. The number of molecules can be a small constant inside the volume. The suction device can tend to recover to a certain greater volume, and therefore the absolute pressure inside the cannula insertion system can tend to drop as the volume increases with the same temperature and number of molecules. As a result, the pressure difference between inside (low pressure) and outside (atmosphere pressure) the suction device tends to maintain the vacuum that generates a suction effect for maintaining the cannula insertion system in a desired position. In some embodiments, the greater the compressible volume, the greater the suction force. In some embodiments, the central cannula in the sclerotomy and the suction cup mutually support each other. In some embodiments, the torque generated by the cannula is counter balanced by the surrounding cup. In some embodiments, the cannula insertion system can be manufactured using low cost injection molding.

### Example Dimensions and Configurations

In some embodiments, the curvature of the contact lid of the cannula insertion sustaining system can be about the same as the average curvature of human sclera.

In some embodiments, the diameter of the contact lid can be less than about 10 mm, which may avoid vacuum leak due to overlap of the contact lid on an uneven boundary of a cornea and sclera during surgery.

In some embodiments, the diameter of the contact lid can be more than about 3 mm, which may provide sufficient retaining vacuum force and counter torque to torque of the cannula tip at the sclerotomy.

In some embodiments, the cannula tip can be located on the geometrical central axis of the cannula insertion sustaining system.

In some embodiments, the volume of the compressible cap, bellow, or similar structure of the cannula insertion sustaining system can be greater than about 20 mm, which may provide sufficient vacuum compared to ambient air pressure.

In some embodiments, the vacuum cap, bellow, or similar structure can be configured not to leak during a surgical or other operation.

In some embodiments, the surface of the contact lid that is in contact with sclera is configured to have an affinity to human tear fluid.

In some embodiments, the cross section of the contact lid can be tapered, which can provide gradient flexibility.

In some embodiments, the cannula can be configured to sustain the force of impact during insertion such that the cannula does not substantially compress or cave in on itself.

In some embodiments, the cannula tubular structure can be configured to allow for 20, 23, 25, or 27 gauge needles, such as a trocar tip or instrument, to pass through the cannula.

In some embodiments, the body of the cannula insertion sustaining system includes an air-compressible part to provide vacuum.

In some embodiments, the body of the cannula insertion sustaining system provides counter torque to the torque of the cannula tip at the sclerotomy from one or more or all possible radial directions.

In some embodiments, the tubular cannula tip comprises, polymer, metallic materials, combinations thereof, and/or the like.

In some embodiments, the compressible cap, bellow, or similar structure of the cannula insertion sustaining system comprises vinyl, silicone, combinations thereof, and/or similar flexible materials.

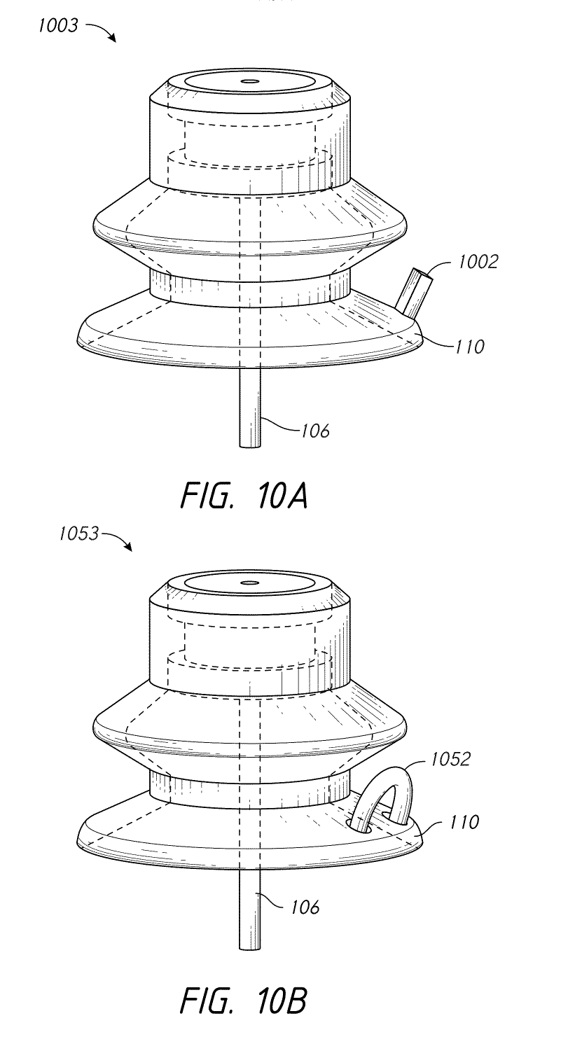

FIG. 10A is a top perspective view of an example of a cannula portion or device 1003, for example for use with a cannula insertion sustaining system (e.g., the system 100), comprising a vacuum release apparatus 1002. In some embodiments, the vacuum release apparatus 1002 is integrally formed with the support structure 110. In some embodiments, the vacuum release apparatus 1002 is separately formed from the support structure 110 and/or is coupled or fixed to the support structure 110. In some embodiments, the vacuum release apparatus 1002 comprises a ring structure for gripping the support structure 110 to release a vacuum seal between the support structure 110 and the sclera. In some embodiments, the vacuum release apparatus 1002 comprises a protrusion portion, an extension, or other mechanism for gripping the support structure 110 to release a vacuum seal between the support structure 110 and the sclera. The vacuum release apparatus 1002 may configured to be flexible or to be non-flexible. In some embodiments, the vacuum release apparatus 1002 comprises any of the materials disclosed herein for the support structure 110, such as, but not limited to, vinyl, silicone, combinations thereof, and/or the like. Any of the foregoing embodiments of the vacuum release apparatus 1002 can be used and combined with each other and/or with any of the cannula insertion sustaining systems disclosed herein.

In some embodiments, a user can release a vacuum between the support structure 110 and the sclera by utilizing tweezers or other surgical tools to grip the vacuum release apparatus 1002. By holding the vacuum release apparatus 1002 and/or pulling away from sclera, the user can create an opening between the support structure 110 and the sclera, thereby allowing air to leak through the lifted lid portion of the support structure 110 and break the vacuum state of the support structure 110.

In some embodiments, a surgeon or other user can use the tip of tweezers or other tools to lift the support structure 110, without using a vacuum release apparatus 1002, to allow air into the support structure 110 and/or to release the vacuum. In this way, vacuum pressure in the support structure 110 can be released to ambient atmospheric pressure.

FIG. 10B is a top perspective view of an example of a cannula portion or device 1053, for example for use with a cannula insertion sustaining system (e.g., the system 100), comprising a vacuum release apparatus 1052. The vacuum release apparatus 1052 comprises an arc shape that may be configured to engage a tool such as tweezers, a hook, etc. without or substantially without a grasping action.

In some embodiments, the cannula insertion sustaining system or a cannula portion comprises an annular flange projecting from the edge of the disk structure. The flange may be engaged as a vacuum release apparatus. An upper surface of the flange may mimic or correspond to a shape of the disk structure, for example being indicative that the disk structure is not properly engaged with the sclera.

While described herein in large part with respect to inserting a cannula in a sclera of an eye, the cannula insertion sustaining systems can also be used with other body parts (e.g., skin, blood vessels, gastrointestinal, combinations thereof, and the like) and other non-medical uses (e.g., transferring material across a membrane in biological or chemical research). Shapes, dimensions, etc. of the disk structure and/or other portions of the systems described herein may be adapted for such uses.

### Example Embodiments

The following example embodiments identify some possible permutations of combinations of features disclosed herein, although other permutations of combinations of features are also possible.

1. A cannula device for an ophthalmic surgical procedure, the device comprising:
a cannula tube configured to be inserted through a sclera of an eye; and
a disk structure configured to apply a force on the sclera to sustain a position of the cannula tube.

2. The cannula device of Embodiment 1, wherein the force comprises at least one of surface tension forces, suction forces, adhesion forces, and vacuum forces.

3. The cannula device of Embodiment 1 or 2, wherein the disk structure comprises a circular disk structure configured to fit a curvature of an eye.

4. The cannula device of Embodiment 1 or 2, wherein the disk structure comprises a circular disk structure curved more than a curvature of an eye.

5. The cannula device of any one of Embodiments 1-4, wherein the disk structure comprises a rim pattern.

6. The cannula device of Embodiment 5, wherein the rim pattern comprises a plurality of protrusions that extend radially outward from an arcuate portion of the disk structure.

7. The cannula device of Embodiment 5 or 6, wherein the rim pattern comprises a surface portion at an outer tip portion of the plurality of protrusions.

8. The cannula device of any one of Embodiments 1-7, further comprising a cannula body portion configured to be above a sclera of an eye.

9. The cannula device of Embodiment 8, wherein the cannula body portion comprises a plunger structure configured to create a vacuum force between the disk structure and a sclera.

10. The cannula device of any one of Embodiments 1-9, further comprising a bellow portion configured to generate a vacuum force between the disk structure and a sclera.

11. The cannula device of any one of Embodiments 1-9, further comprising a stepped cup configured to generate a vacuum force between the disk structure and a sclera.

12. The cannula device of any one of Embodiments 1-11, further comprising a vacuum release apparatus.

13. A cannula insertion sustaining system for an ophthalmic procedure, the system comprising:
the cannula device of any one of Embodiments 1-12;
a connector tube coupled to the cannula tube, the connector tube configured to reduce an effective pulling force on the cannula tube.

14. A cannula insertion sustaining system for an ophthalmic procedure, the system comprising:
a cannula tube configured to be inserted through a sclera of an eye;
an upper tube portion; and
a connector tube coupled to the upper tube portion and to the cannula tube, the connector tube configured to reduce an effective pulling vector force asserted on the cannula tube.

15. The cannula insertion sustaining system of Embodiment 14, wherein the connector tube is flexible to enable the connector tube to be curved.

16. The cannula insertion sustaining system of Embodiment 14, wherein the connector tube is flexible to enable the connector tube to be curved up to an angle.

17. The cannula insertion sustaining system of any one of Embodiments 14-16, wherein the connector tube is curved at an angle.

18. The cannula insertion sustaining system of any one of Embodiments 14-17, further comprising a plunger structure configured to create a vacuum force for sustaining a position of the cannula.

19. The cannula insertion sustaining system of any one of Embodiments 14-18, further comprising a support structure configured to apply a force on the sclera, the force configured to enable the support structure to sustain a position of the cannula tube.

20. The cannula insertion sustaining system of Embodiment 19, wherein the force comprises at least one of surface tension forces, suction forces, adhesion forces, and vacuum forces.

21. The cannula insertion sustaining system of Embodiment 19 or 20, wherein the support structure comprises a disk structure.

22. A method of sustaining a position of a cannula in a sclera, the method comprising:
inserting the cannula through the sclera; and
engaging a support structure with the sclera to apply a force on the sclera.

23. The method of Embodiment 22, wherein inserting the cannula through the sclera comprises inserting a trocar through the sclera, the cannula around the trocar.

24. The method of Embodiment 22 or 23, further comprising actuating a plunger structure to create a vacuum between the support structure and the sclera.

25. The method of any one of Embodiments 22-24, further comprising actuating a bellow portion to create a vacuum between the support structure and the sclera.

26. The method of any one of Embodiments 22-25, wherein the support structure comprises a vacuum release apparatus, and wherein the method comprises engaging the vacuum release apparatus to release at least part of the force on the sclera.

27. The method of any one of Embodiments 22-26, further comprising inserting a fluid through the cannula.

28. The method of any one of Embodiments 22-27, wherein the cannula is coupled to a tube connector, wherein the method comprises applying a pulling force to the cannula, and wherein the tube connector reduces an effect of the pulling force on the cannula.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Additionally, the skilled artisan will recognize that any of the above-described methods can be carried out using any appropriate apparatus. Further, the disclosure herein of any particular feature, aspect, method, property, characteristic, quality, attribute, element, or the like in connection with an embodiment can be used in all other embodiments set forth herein. For all of the embodiments described herein the steps of the methods need not be performed sequentially. Thus, it is intended that the scope of the present invention herein disclosed should not be limited by the particular disclosed embodiments described above.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. For example, actions such as “inserting a cannula tube in a sclera” include “instructing insertion of a cannula tube in a sclera.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 45°” includes “45°.” Phrases preceded by a term such as “substantially” include the recited phrase and should be interpreted based on the circumstances (e.g., as much as reasonably possible under the circumstances). For example, “substantially nonflexible” includes “nonflexible.”

What is claimed is:

1. A cannula device for an ophthalmic surgical procedure, the device comprising:
a cannula configured to be inserted through a sclera of an eye; and
a disk structure configured to apply a force on the sclera to sustain a position of the cannula.

2. The cannula device of claim 1, wherein the force comprises at least one of surface tension forces, suction forces, adhesion forces, and vacuum forces.

3. The cannula device of claim 1, wherein the disk structure comprises a circular disk structure configured to fit a curvature of an eye.

4. The cannula device of claim 1, wherein the disk structure comprises a circular disk structure curved more than a curvature of an eye.

5. The cannula device of claim 1, wherein the disk structure comprises a rim pattern.

6. The cannula device of claim 5, wherein the rim pattern comprises a plurality of protrusions that extend radially outward from an arcuate portion of the disk structure.

7. The cannula device of claim 5, wherein the rim pattern comprises a surface portion at an outer tip portion of the plurality of protrusions.

8. The cannula device of claim 1, further comprising a cannula body portion configured to be above a sclera of an eye.

9. The cannula device of claim 8, wherein the cannula body portion comprises a plunger structure configured to create a vacuum force between the disk structure and a sclera.

10. The cannula device of claim 1, further comprising a bellow portion configured to generate a vacuum force between the disk structure and a sclera.

11. The cannula device of claim 1, further comprising a stepped cup configured to generate a vacuum force between the disk structure and a sclera.

12. The cannula device of claim 1, further comprising a vacuum release apparatus.

13. A cannula insertion sustaining system for an ophthalmic procedure, the system comprising:
the cannula device of claim 1;
a connector tube coupled to the cannula, the connector tube configured to reduce an effective pulling force on the cannula.

14. A cannula insertion sustaining system for an ophthalmic procedure, the system comprising:
a cannula tube configured to be inserted through a sclera of an eye;
an upper tube portion; and
a connector tube coupled to the upper tube portion and to the cannula tube, the connector tube configured to reduce an effective pulling vector force asserted on the cannula tube.

15. The cannula insertion sustaining system of claim 14, wherein the connector tube is flexible to enable the connector tube to be curved.

16. The cannula insertion sustaining system of claim 14, wherein the connector tube is flexible to enable the connector tube to be curved up to an angle.

17. The cannula insertion sustaining system of claim 14, wherein the connector tube is curved at an angle.

18. The cannula insertion sustaining system of claim 14, further comprising a plunger structure configured to create a vacuum force for sustaining a position of the cannula.

19. The cannula insertion sustaining system of claim 14, further comprising a support structure configured to apply a force on the sclera, the force configured to enable the support structure to sustain a position of the cannula tube.

20. The cannula insertion sustaining system of claim 19, wherein the force comprises at least one of surface tension forces, suction forces, adhesion forces, and vacuum forces.

21. The cannula insertion sustaining system of claim 19, wherein the support structure comprises a disk structure.
